(19) United States
(12) Patent Application Publication
(10) Pub. No.: US 2022/0062528 A1
(43) Pub. Date: Mar. 3, 2022

(54) Aspiration Syringes and Methods Thereof

(71) Applicant: Bard Access Systems, Inc., Salt Lake City, UT (US)

(72) Inventors: Kyle G. Thornley, Farmington, UT (US); Bryan Stone, Fruit Heights, UT (US)

(21) Appl. No.: 17/461,619

(22) Filed: Aug. 30, 2021

Related U.S. Application Data
(60) Provisional application No. 63/072,711, filed on Aug. 31, 2020.

(51) Int. Cl. A61M 1/00; A61M 39/02

(52) U.S. Cl. CPC A61M 1/815; A61M 39/0247; A61M 2202/0413; A61M 2039/0291; A61M 2039/0258

(57) ABSTRACT

Aspiration syringes and methods thereof are disclosed. An exemplary aspiration syringe can include a barrel, a plunger disposed in the barrel, and an aspiration mechanism. The aspiration mechanism can include a thumb-support member coupled to a distal portion of the barrel and a syringe housing slidably disposed around the barrel. The syringe housing can include a proximal portion coupled to a proximal portion of the plunger and a distal portion terminating with a flange incorporated into a finger-support member. The aspiration mechanism can be configured for withdrawing the plunger from the barrel as the finger-support member is slid over the barrel toward the thumb-support member. An example method of the foregoing aspiration syringe can include a fluid-aspirating step including aspirating fluid from a fluid-containing space with a single hand over a medial portion of the syringe by squeezing together the finger-support member and the thumb-support member.

### PRIORITY

This application claims the benefit of priority to U.S. Provisional Application No. 63/072,711, filed Aug. 31, 2020, which is incorporated by reference in its entirety into this application.

### BACKGROUND

Traditional syringes were primarily designed for infusion of fluids. However, such syringes are commonly used to draw a slight vacuum during blood-vessel cannulation. The vacuum provides a clinician a means for receiving tactile feedback upon successfully establishing blood-vessel access, the feedback resulting from a pressure differential upon release of the vacuum when a tip of a needle coupled to the syringe enters the blood vessel. With traditional syringes, there is a lack of support for the clinician to both secure the syringe and draw the vacuum while inserting the needle into the blood vessel. What is needed is an aspiration syringe that is specifically configured with at least the foregoing support for blood-vessel cannulation.

Disclosed herein are aspiration syringes and methods thereof.

### SUMMARY

Disclosed herein is an aspiration syringe including, in some embodiments, a barrel, a plunger disposed in the barrel, and an aspiration mechanism. The aspiration mechanism includes a thumb-support member coupled to a proximal portion of the barrel and a syringe housing slidably disposed around the barrel. The syringe housing includes a proximal portion coupled to a proximal portion of the plunger and a distal portion terminating with a flange incorporated into a finger-support member. The aspiration mechanism is configured for withdrawing the plunger from the barrel as the finger-support member is slid over the barrel toward the thumb-support member.

In some embodiments, the syringe housing and the plunger are molded together in an integral piece such that the proximal portion of the syringe housing is integral with the proximal portion of the plunger.

In some embodiments, the syringe housing and the plunger are separately molded pieces coupled together in a coupled piece such that the proximal portion of the syringe housing is bonded or adhered to the proximal portion of the plunger.

In some embodiments, the syringe housing includes a longitudinal gap within which the thumb-support member moves relative to the syringe housing when the syringe housing is slid over the barrel.

In some embodiments, the flange includes a pair of finger recesses in a distal face of the flange. The pair of finger recesses is configured to respectively cradle a pair of finger pads of a pair of fingers of the single hand. The pair of finger recesses is also configured to encourage placement of each finger of the pair of fingers on an opposite side of the barrel.

In some embodiments, the flange is annular such that the flange encircles the barrel. Alternatively, the flange is coupled to a distal annular member with the distal annular member encircling the barrel. The flange or the distal annular member is configured to accommodate a pair of finger pads of a pair of fingers of the single hand placed against a distal side thereof in any chosen placement of each finger of the pair of fingers around the flange or the distal annular member.

In some embodiments, the flange or the distal annular member includes an annular finger recess in a distal face thereof. The annular finger recess is configured to cradle the pair of finger pads of the pair of fingers.

In some embodiments, the thumb-support member includes a single thumb recess in a proximal face of the thumb-support member. The thumb recess is configured to cradle a thumbpad of a thumb. The thumb recess is also configured to encourage approximately longitudinal alignment of the thumb with a surface normal of a surface of the barrel.

In some embodiments, the thumb-support member is coupled to or formed with a proximal annular member with the proximal annular member encircling the barrel. The proximal annular member is configured to accommodate a thumbpad of a thumb placed against a proximal side thereof in any chosen placement of the thumb around the proximal annular member.

In some embodiments, the proximal annular member includes an annular thumb recess in a proximal face thereof. The annular thumb recess is configured to cradle the thumbpad of the thumb.

In some embodiments, a distal portion of the barrel terminates with a Luer-tapered syringe tip.

In some embodiments, the distal portion of the barrel further terminates with an internally threaded connector around the syringe tip. The internally threaded connector is configured to screw together with an externally threaded or flanged connector of another medical device.

Also disclosed herein is a method of cannulating with an aspiration syringe. The method includes an obtaining step, a needle-inserting step, and a vacuum-drawing step. The obtaining step includes obtaining the aspiration syringe. The aspiration syringe includes a thumb-support member coupled to a proximal portion of a barrel and a syringe housing slidably disposed around the barrel. The syringe housing includes a proximal portion coupled to a proximal portion of a plunger, as well as a distal portion terminating with a flange incorporated into a finger-support member. The needle-inserting step includes inserting a needle coupled to the aspiration syringe into subcutaneous tissue of a patient. The vacuum-drawing step includes drawing a vacuum with the aspiration syringe while inserting the needle into the subcutaneous tissue of the patient. The vacuum-drawing step is performed with a single hand over a medial portion of the aspiration syringe by squeezing together the finger-support member and the thumb-support member. Squeezing together the finger-support member and the thumb-support member proximally slides the syringe housing over the barrel and withdraws the plunger from the barrel.

In some embodiments, the method further includes a needle-coupling step. The needle-coupling step includes coupling a needle to the aspiration syringe, which, in turn, includes screwing a flanged connector of a needle hub into an internally threaded connector around a syringe tip of the aspiration syringe.

In some embodiments, the squeezing together of the finger-support member and the thumb-support member during the vacuum-drawing step includes moving the thumb-support member through a longitudinal gap within the syringe housing.

In some embodiments, the method further includes a finger-placing step. The finger-placing step includes placing a pair of finger pads of a pair of fingers of the single hand into a pair of finger recesses in a distal face of the flange on opposite sides of the barrel. The finger-placing step is performed before the vacuum-drawing step.

In some embodiments, an alternative of the finger-placing step includes placing the pair of finger pads of the pair of fingers of the single hand anywhere against a distal side of the flange when the flange is annular. When the flange is coupled to a distal annular member, the finger-placing step includes placing the pair of finger pads of the pair of fingers of the single hand anywhere against a distal side of the distal annular member. Again, the finger-placing step is performed before the vacuum-drawing step.

In some embodiments, the alternative of the finger-placing step includes placing the pair of finger pads in an annular finger recess in a distal face of the flange or the distal annular member.

In some embodiments, the method further includes a thumb-placing step. The thumb-placing step includes placing a thumbpad of a thumb of the single hand into a single thumb recess in a proximal face of the thumb-support member with the thumb approximately longitudinally aligned with a surface normal of a surface of the barrel. The finger-placing step is performed before the vacuum-drawing step.

In some embodiments, an alternative of the thumb-placing step includes placing the thumbpad of the thumb of the single hand anywhere against a proximal side of a proximal annular member coupled to or formed with the thumb-support member. Again, the thumb-placing step is performed before the vacuum-drawing step.

In some embodiments, the alternative of the thumb-placing step includes placing the thumbpad in an annular thumb recess in a proximal face of the proximal annular member.

In some embodiments, the method further includes a needle tract-establishing step. The needle tract-establishing step includes establishing a needle tract from a skin surface of the patient to a fluid-containing space within the patient. Establishment of the needle tract is realized upon receiving tactile feedback resulting from a pressure differential occurring with a release of the vacuum when a tip of the needle enters the fluid-containing space.

In some embodiments, the fluid-containing space is a blood-vessel lumen including blood as a fluid therein.

Also disclosed herein is a method of aspirating a fluid with an aspiration syringe. The method includes an obtaining step, a needle tract-establishing step, and a fluid-aspirating step. The obtaining step includes obtaining the aspiration syringe. The aspiration syringe includes a thumb-support member coupled to a proximal portion of a barrel and a syringe housing slidably disposed around the barrel. The syringe housing includes a proximal portion coupled to a proximal portion of a plunger, as well as a distal portion terminating with a flange incorporated into a finger-support member. The needle tract-establishing step includes establishing a needle tract with a needle coupled to the aspiration syringe from a skin surface to a fluid-containing space within a patient. The fluid-aspirating step includes aspirating fluid from the fluid-containing space with a single hand over a medial portion of the aspiration syringe by squeezing together the finger-support member and the thumb-support member. Squeezing together the finger-support member and the thumb-support member proximally slides the syringe housing over the barrel and withdraws the plunger from the barrel.

In some embodiments, the method further includes a needle-coupling step. The needle-coupling step includes coupling a needle to the aspiration syringe, which, in turn, includes screwing a flanged connector of a needle hub into an internally threaded connector around a syringe tip of the aspiration syringe.

In some embodiments, the squeezing together of the finger-support member and the thumb-support member during the fluid-aspirating step includes moving the thumb-support member through a longitudinal gap within the syringe housing.

In some embodiments, the method further includes a finger-placing step. The finger-placing step includes placing a pair of finger pads of a pair of fingers of the single hand into a pair of finger recesses in a distal face of the flange on opposite sides of the barrel. The finger-placing step is performed before the fluid-aspirating step.

In some embodiments, an alternative of the finger-placing step includes placing the pair of finger pads of the pair of fingers of the single hand anywhere against a distal side of the flange when the flange is annular. When the flange is coupled to a distal annular member, the finger-placing step includes placing the pair of finger pads of the pair of fingers of the single hand anywhere against a distal side of the distal annular member. Again, the finger-placing step is performed before the fluid-aspirating step.

In some embodiments, the alternative of the finger-placing step includes placing the pair of finger pads in an annular finger recess in a distal face of the flange or the distal annular member.

In some embodiments, the method further includes a thumb-placing step. The thumb-placing step includes placing a thumbpad of a thumb of the single hand into a single thumb recess in a proximal face of the thumb-support member with the thumb approximately longitudinally aligned with a surface normal of a surface of the barrel. The finger-placing step is performed before the fluid-aspirating step.

In some embodiments, an alternative of the thumb-placing step includes placing the thumbpad of the thumb of the single hand anywhere against a proximal side of a proximal annular member coupled to or formed with the thumb-support member. Again, the thumb-placing step is performed before the fluid-aspirating step.

In some embodiments, the alternative of the thumb-placing step includes placing the thumbpad in an annular thumb recess in a proximal face of the proximal annular member.

In some embodiments, the fluid-containing space is a blood-vessel lumen and the fluid is blood.

These and other features of the concepts provided herein will become more apparent to those of skill in the art in view of the accompanying drawings and following description, which describe particular embodiments of such concepts in greater detail.

### DRAWINGS

FIG. 1 illustrates a perspective view of an aspiration syringe in a ready-to-aspirate state in accordance with some embodiments.

FIG. 2 illustrates a perspective view of the aspiration syringe after aspiration of a volume of a fluid in accordance with some embodiments.

FIG. 3 illustrates a top view of the aspiration syringe of FIG. 1 in accordance with some embodiments.

FIG. 4 illustrates a top view of the aspiration syringe of FIG. 2 in accordance with some embodiments.

FIG. 5 illustrates a perspective view of another aspiration syringe in a ready-to-aspirate state in accordance with some embodiments.

FIG. 6 illustrates a perspective view of the other aspiration syringe after aspiration of a volume of a fluid in accordance with some embodiments.

FIG. 7 illustrates a top view of the aspiration syringe of FIG. 5 in accordance with some embodiments.

FIG. 8 illustrates a top view of the aspiration syringe of FIG. 6 in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal-end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal-end portion, or the proximal length of the catheter can include the proximal end of the catheter; however, the proximal portion, the proximal-end portion, or the proximal length of the catheter need not include the proximal end of the catheter. That is, unless context suggests otherwise, the proximal portion, the proximal-end portion, or the proximal length of the catheter is not a terminal portion or terminal length of the catheter.

With respect to “distal,” a “distal portion” or a “distal-end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal-end portion, or the distal length of the catheter can include the distal end of the catheter; however, the distal portion, the distal-end portion, or the distal length of the catheter need not include the distal end of the catheter. That is, unless context suggests otherwise, the distal portion, the distal-end portion, or the distal length of the catheter is not a terminal portion or terminal length of the catheter.

Unless defined otherwise, all technical and scientific terms used herein have the same meaning as commonly understood by those of ordinary skill in the art.

As set forth above, an aspiration syringe is needed that is specifically configured for a clinician to both secure the aspiration syringe and draw a vacuum during blood-vessel cannulation for receiving tactile feedback upon successfully establishing blood-vessel access. Disclosed herein are aspiration syringes and methods thereof.

### Aspiration Syringes

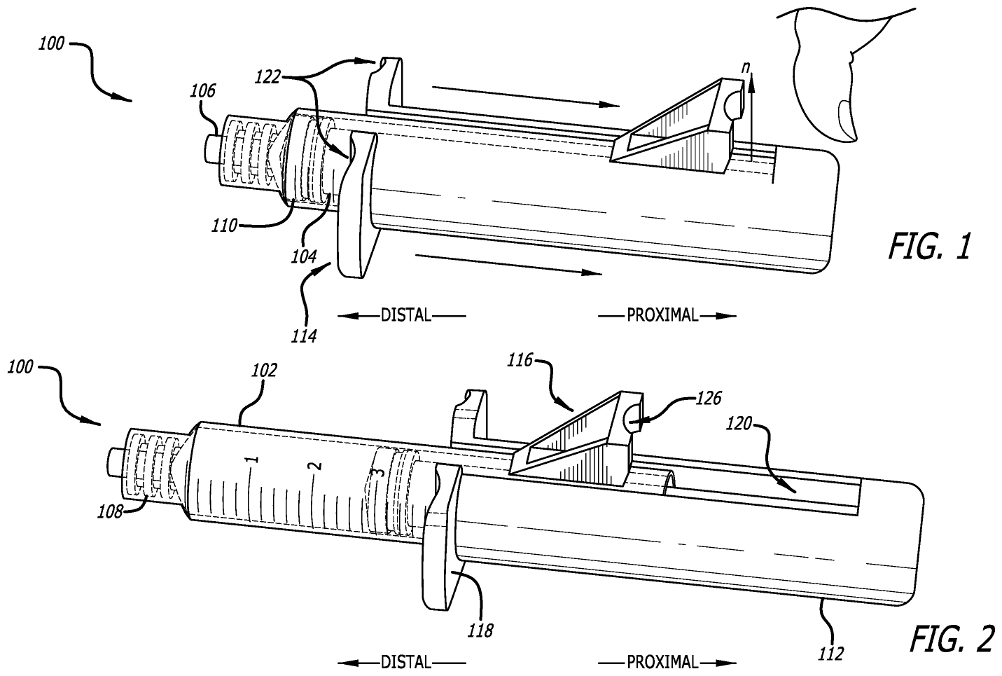

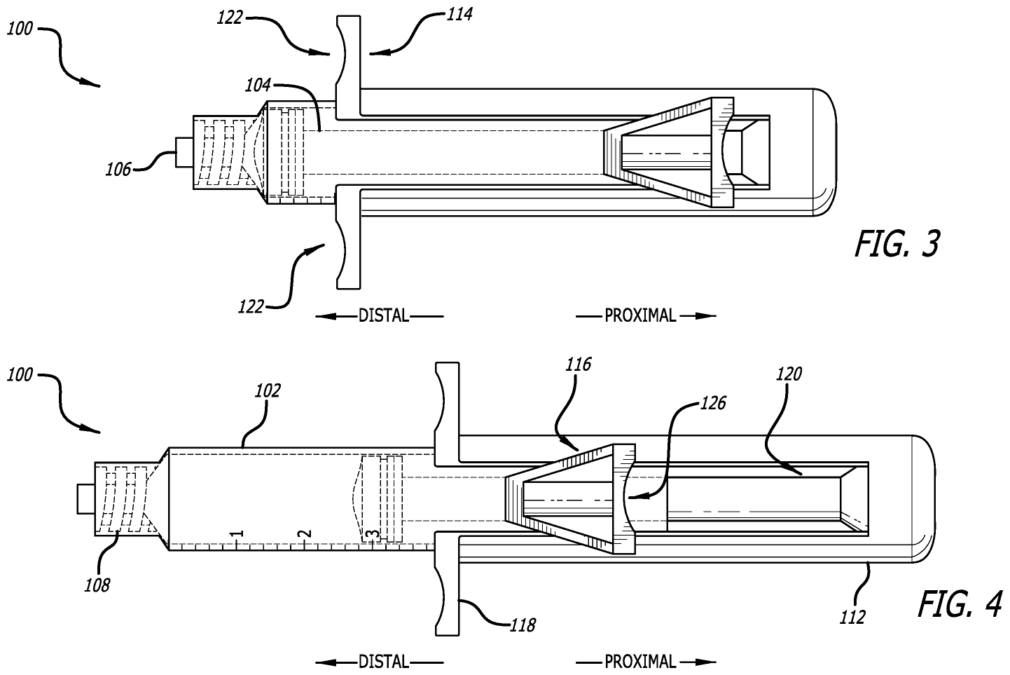

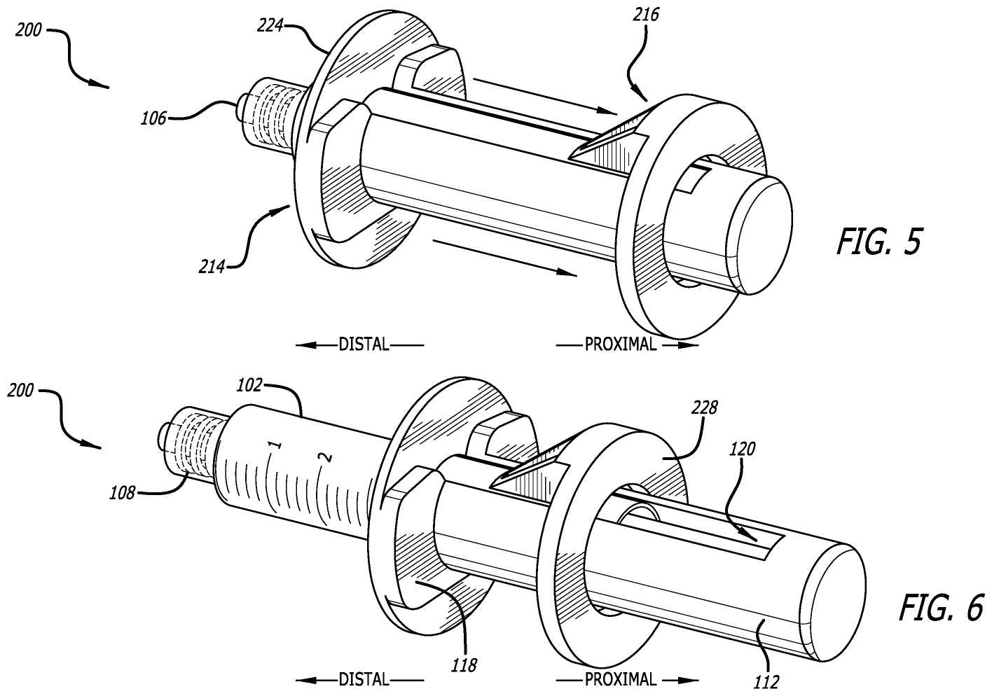

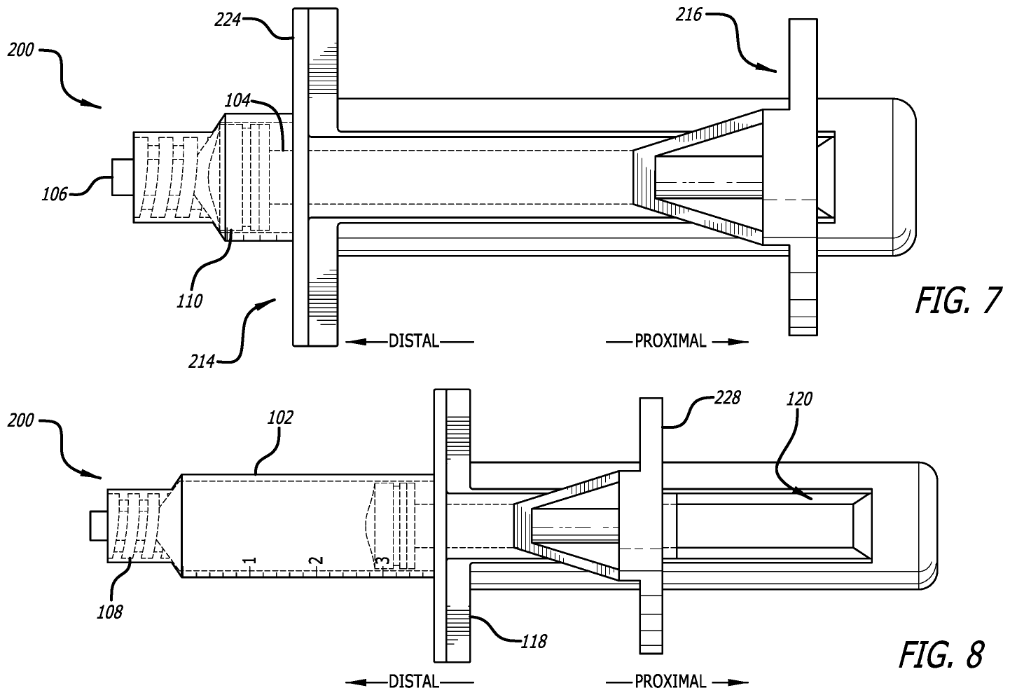

FIGS. 1-4 and FIGS. 5-8 illustrate two different views and two different states for two aspiration syringes in accordance with some embodiments. FIGS. 1 and 3 illustrate an aspiration syringe 100 in a ready-to-aspirate state, while FIGS. 2 and 4 illustrate the aspiration syringe 100 after aspiration of a volume of a fluid. Likewise, FIGS. 5 and 7 illustrate an aspiration syringe 200 in a ready-to-aspirate state, while FIGS. 6 and 8 illustrate the aspiration syringe 200 after aspiration of a volume of a fluid. In FIGS. 2, 4, 6, and 8 the fluid is merely air; however, the fluid can be a bodily fluid such as blood as set forth in the methods below with respect to at least the fluid-aspirating step.

As shown, the aspiration syringe 100 or 200 includes a barrel 102, a plunger 104 disposed in the barrel 102, and an aspiration mechanism.

The barrel 102 includes a proximal portion terminating with a proximal opening (not shown) through which the plunger 104 is disposed. The barrel 102 also includes a distal portion terminating with a Luer-tapered syringe tip 106 having a distal opening, the syringe tip 106 configured to insert into a Luer-tapered receptacle of another medical device (e.g., a needle including a needle hub having the receptacle). The distal portion of the barrel 102 further terminates with an internally threaded connector 108 (e.g., a collar) around the syringe tip 106. The internally threaded connector 108 is configured to screw together with an externally threaded or flanged connector of the other medical device (e.g., the needle including the needle hub having the flanged connector).

The barrel 102 further includes graduations printed or molded onto the barrel 102 for determining how much fluid is aspirated into the aspiration syringe 100 or 200 when the plunger 104 is withdrawn from its initial position in the barrel 102 in the ready-to-aspirate state of the aspiration syringe 100 or 200.

The plunger 104 includes a proximal portion fixedly coupled to the syringe housing 112 as set forth below with respect to the syringe housing 112. The plunger 104 also includes a distal portion terminating with a gasket 110, the gasket 110 configured to fluidly seal off adjoining portions of the barrel 102 from each other, particularly upon aspiration of a volume of a fluid with the aspiration syringe 100 or 200.

The aspiration mechanism includes a syringe housing 112 slidably disposed around the barrel 102, a finger-support member 114 or 214, and a thumb-support member 116 or 216. The aspiration mechanism is configured for withdrawing the plunger 104 from the barrel 102 as the syringe housing 112 is slid over the barrel 102 and the finger-support member 114 or 214 is brought toward the thumb-support member 116 or 216 such as by squeezing the finger-support member 114 or 214 and thumb-support member 116 or 216 together.

The syringe housing 112 includes a proximal portion fixedly coupled to a proximal portion of the plunger 104 and a distal portion terminating with a flange 118 incorporated into the finger-support member 114 or 214. The syringe housing 112 and the plunger 104 can be separately molded pieces coupled together in a coupled piece such that the proximal portion of the syringe housing 112 is adhered or bonded (e.g., solvent bonded) to the proximal portion of the plunger 104. Alternatively, the syringe housing 112 and the plunger 104 can be molded together in an integral piece such that the proximal portion of the syringe housing 112 is integral with the proximal portion of the plunger 104.

The syringe housing 112 includes a longitudinal gap 120 within which the thumb-support member 116 or 216 moves when the syringe housing 112 is slid over the barrel 102 (e.g., by squeezing the finger-support member 114 or 214 and thumb-support member 116 or 216 together). As set forth below, the thumb-support member 116 or 216 is fixedly coupled to the barrel 102, which restricts the thumb-support member 116 or 216 from moving. Indeed, it is the syringe housing 112 that moves as it is proximally slid over the barrel 102. When the syringe housing 112 is the frame of reference, however, the thumb-support member 116 or 216 moves relative to the syringe housing 112 in the longitudinal gap 120.

The finger-support member 114 or 214 incorporates the flange 118 of the syringe housing 112. Description for each finger-support member of the finger-support members 114 and 214 is set forth below. It should be understood that while the finger-support member 114 is shown to be part of the aspiration syringe 100 shown in FIGS. 1-4, the finger-support member 114 can alternatively be part of the aspiration syringe 200 shown in FIGS. 5-8. Likewise, while the finger-support member 214 is shown to be part of the aspiration syringe 200 shown in FIGS. 5-8, the finger-support member 214 can alternatively be part of the aspiration syringe 100 shown in FIGS. 1-4.

The finger-support member 114 includes a pair of finger recesses 122 in a distal face of the flange 118. The pair of finger recesses 122 is configured to respectively cradle a pair of finger pads of a pair of fingers of a single hand. The pair of finger recesses 122 is also configured to encourage placement of each finger of the pair of fingers on an opposite side of the barrel 102.

The finger-support member 214 is a distal annular member 224 fixedly coupled to the flange 118. Alternatively, the finger-support member 214 is formed together with the flange 118 when the flange 118 is annular. Regardless, the distal annular member 224 or the flange 118, when annular, encircles the barrel 102 such that the barrel 102 passes through a central hole of the distal annular member 224 or the flange 118. When the distal annular member 224 is coupled to the flange 118, the distal annular member 224 and the flange 118 can be separately molded pieces coupled (e.g., adhered, solvent bonded, etc.) together in a coupled piece. Alternatively, the distal annular member 224 and the flange 118 can be molded together in an integral piece. Like the finger-support member 114, the distal annular member 224 or the flange 118, when annular, is configured to accommodate a pair of finger pads of a pair of fingers of a single hand placed against a distal side thereof; however, the distal annular member 224 or the flange 118 is configured to encourage placement of the pair of finger against the distal side of the distal annular member 224 or the flange 118 in any desired placement of the pair of fingers around the distal annular member 224 or the flange 118. While not shown, the distal annular member 224 can include an annular finger recess in a distal face thereof. The annular finger recess is configured to cradle the pair of finger pads of the pair of fingers.

The thumb-support member 116 or 216 is fixedly coupled (e.g., adhered, solvent bonded, etc.) to and extends from the proximal portion of the barrel 102. Description for each thumb-support member of the thumb-support members 116 and 216 is set forth below. It should be understood that while the thumb-support member 116 is shown to be part of the aspiration syringe 100 shown in FIGS. 1-4, the thumb-support member 100 can alternatively be part of the aspiration syringe 200 shown in FIGS. 5-8. Likewise, while the thumb-support member 216 is shown to be part of the aspiration syringe 200 shown in FIGS. 5-8, the thumb-support member 216 can alternatively be part of the aspiration syringe 100 shown in FIGS. 1-4.

The thumb-support member 116 includes a single thumb recess 126 in a proximal face of the thumb-support member 116. The thumb recess 126 is configured to cradle a thumbpad of a thumb of a same hand as the pair of fingers used on the finger-support member 114 or 214 for three-finger aspiration of the aspiration syringe 100 with a natural grip of the hand. The thumb recess 126 is also configured to encourage approximately longitudinal alignment of the thumb with a surface normal of a surface of the barrel 102 as shown in FIG. 1.

The thumb-support member 216 is a proximal annular member 228 fixedly coupled to the thumb-support member 116. Alternatively, the thumb-support member 216 is formed together with the proximal annular member 228 in that merely the proximal annular member 228 is fixedly coupled to the proximal portion of the barrel 102. Regardless, the proximal annular member 228 encircles the barrel 102 such that the barrel 102 passes through a central hole of the proximal annular member 228. Indeed, the thumb-support member 216 and the proximal annular member 228 can be separately molded pieces coupled (e.g., adhered, solvent bonded, etc.) together in a coupled piece. Alternatively, the thumb-support member 216 and the proximal annular member 228 can be molded together in an integral piece. Like the thumb-support member 116, the proximal annular member 228 is configured to accommodate a thumbpad of a thumb of a same hand as the pair of fingers used on the finger-support member 114 or 214 placed against a proximal side thereof; however, the proximal annular member 228 is configured to encourage placement of the thumb against the proximal side of the proximal annular member 228 in any desired placement of the thumb around the proximal annular member 228 for three-finger aspiration of the aspiration syringe 200 with a natural grip of the hand. While not shown, the proximal annular member 228 can include an annular thumb recess in a proximal face thereof. The annular thumb recess is configured to cradle the thumbpad of the thumb.

### Methods

Methods of the aspiration syringes 100 and 200 include methods of cannulating, as well as methods of aspirating with the aspiration syringes 100 and 200. Being as cannulation of, for example, a fluid-containing space such as a blood-vessel lumen occurs before aspiration of fluid such as blood therefrom, a method of cannulating with the aspiration syringe 100 or 200 is set forth below including steps for aspirating with the aspiration syringe 100 or 200.

The method of cannulating with the aspiration syringe 100 or 200 includes an obtaining step, a needle-inserting step, and a vacuum-drawing step.

The obtaining step includes obtaining the aspiration syringe 100 or 200. As set forth above, the aspiration syringe 100 or 200 includes the thumb-support member 116 or 216 coupled to the distal portion of the barrel 102 and the syringe housing 112 slidably disposed around the barrel 102. The syringe housing 112 includes the proximal portion coupled to the proximal portion of the plunger 104, as well as the distal portion terminating with the flange 118 incorporated into the finger-support member 114 or 214.

Before the needle-inserting step, the method can further include a needle-coupling step. The needle-coupling step includes coupling a needle to the aspiration syringe 100 or 200, which, in turn, includes screwing a flanged connector of a needle hub into the internally threaded connector 108 around the syringe tip 106 of the aspiration syringe 100 or 200.

The needle-inserting step includes inserting the needle coupled to the aspiration syringe 100 or 200 into subcutaneous tissue of a patient.

Before the vacuum-drawing step, the method further includes a finger-placing step. With respect to the aspiration syringe 100, the finger-placing step includes placing a pair of finger pads of a pair of fingers of the single hand into the pair of finger recesses 122 in the distal face of the flange 118 on opposite sides of the barrel 102. When aspirating with the aspiration syringe 100 as set forth below, the finger-placing step is performed before the fluid-aspirating step if the single hand is partially or wholly removed from the aspiration syringe 100 after any intervening steps between cannulating and aspirating with the aspiration syringe 100.

An alternative of the foregoing finger-placing step is performed for the aspiration syringe 200 or any like syringe including an annular flange or the distal annular member 224. Such an alternative finger-placing step includes placing the pair of finger pads of the pair of fingers of the single hand anywhere against the distal side of the flange 118 when the flange 118 is annular. When the flange 118 is coupled to the distal annular member 224, the finger-placing step includes placing the pair of finger pads of the pair of fingers of the single hand anywhere against the distal side of the distal annular member 224. For example, the alternative of the finger-placing step can include placing the pair of finger pads in the annular finger recess in the distal face of the flange 118 or the distal annular member 224. Like the foregoing finger-placing step, the alternative finger-placing step is performed before the vacuum-drawing step. When aspirating with the syringe 200 as set forth below, the alternative finger-placing step is performed before the fluid-aspirating step if the single hand is partially or wholly removed from the aspiration syringe 200 after any intervening steps between cannulating and aspirating with the aspiration syringe 200.

Further before the vacuum-drawing step, the method further includes a thumb-placing step. With respect to the aspiration syringe 100, the thumb-placing step includes placing a thumbpad of a thumb of the single hand into the single thumb recess 126 in the proximal face of the thumb-support member 116 with the thumb approximately longitudinally aligned with a surface normal of the surface of the barrel 102. When aspirating with the aspiration syringe 100 as set forth below, the finger-placing step is performed before the fluid-aspirating step if the single hand is partially or wholly removed from the aspiration syringe 100 after any intervening steps between cannulating and aspirating with the aspiration syringe 100.

An alternative of the foregoing thumb-placing step is performed for the aspiration syringe 200 or any like syringe including the proximal annular member 228. Such an alternative thumb-placing step includes placing the thumbpad of the thumb of the single hand anywhere against the proximal side of the proximal annular member 228 coupled to or formed with the thumb-support member 216. For example, the alternative of the thumb-placing step can include placing the thumbpad in the annular thumb recess in the proximal face of the proximal annular member 228. Like the foregoing thumb-placing step, the alternative thumb-placing step is performed before the vacuum-drawing step. When aspirating with the syringe 200 as set forth below, the alternative thumb-placing step is performed before the fluid-aspirating step if the single hand is partially or wholly removed from the aspiration syringe 200 after any intervening steps between cannulating and aspirating with the aspiration syringe 200.

The vacuum-drawing step includes drawing a vacuum with the aspiration syringe 100 or 200 while inserting the needle into the subcutaneous tissue of the patient such as while actively performing the needle-inserting step or pausing somewhere in a middle of the needle-inserting step to perform the vacuum-drawing step. The vacuum-drawing step is performed with the single hand over, under, or to a side of a medial portion of the aspiration syringe 100 or 200 by squeezing together the finger-support member 114 or 214 and the thumb-support member 116 or 216. Squeezing together the finger-support member 114 or 214 and the thumb-support member 116 or 216 proximally slides the syringe housing 112 over the barrel 102 and withdraws the plunger 104 from the barrel 102 creating the vacuum. The squeezing together of the finger-support member 114 or 214 and the thumb-support member 116 or 216 during the vacuum-drawing step includes moving the thumb-support member 116 or 216 through the longitudinal gap 120 within the syringe housing 112. Again, it should be understood that the thumb-support member 116 or 216 is coupled to the barrel 102, and, as such, the thumb-support member 116 or 216 does not actually move. Indeed, it is the syringe housing 112 that moves as it is proximally slid over the barrel 102.

The method further includes a needle tract-establishing step. The needle tract-establishing step includes establishing a needle tract from a skin surface of the patient to the fluid-containing space within the patient. Establishment of the needle tract is realized upon receiving tactile feedback resulting from a pressure differential occurring with a release of the vacuum created during the vacuum-drawing step when a tip of the needle enters the fluid-containing space.

Upon establishing the needle tract in accordance with the needle tract-establishing step, fluid such as blood from the fluid-containing space such as a blood-vessel lumen can be aspirated with the aspiration syringe 100 or 200 in a fluid-aspirating step. The fluid-aspirating step includes aspirating the fluid from the fluid-containing space with a same or different hand as the foregoing single hand over, under, or to a side of the medial portion of the aspiration syringe 100 or 200 by squeezing together the finger-support member 114 or 214 and the thumb-support member 116 or 216. Squeezing together the finger-support member 114 or 214 and the thumb-support member 116 or 216 proximally slides the syringe housing 112 over the barrel 102 and withdraws the plunger 104 from the barrel 102 creating a vacuum. The squeezing together of the finger-support member 114 or 214 and the thumb-support member 116 or 216 during the fluid-aspirating step includes moving the thumb-support member 116 or 216 through the longitudinal gap 120 within the syringe housing 112. Again, it should be understood that the thumb-support member 116 or 216 is coupled to the barrel 102, and, as such, the thumb-support member 116 or 216 does not actually move. Indeed, it is the syringe housing 112 that moves as it is proximally slid over the barrel 102.

It should be understood that while the aspiration syringes 100 and 200 are designed for three-finger aspiration and the like, some clinicians might choose to use two fingers or up to all five finger of a single, which is within the scope of the concepts provided herein.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. An aspiration syringe, comprising:
a barrel;
a plunger disposed in the barrel; and
an aspiration mechanism including:
a thumb-support member coupled to a proximal portion of the barrel; and
a syringe housing slidably disposed around the barrel including a proximal portion coupled to a proximal portion of the plunger and a distal portion terminating with a flange incorporated into a finger-support member, the aspiration mechanism configured for withdrawing the plunger from the barrel as the finger-support member is slid over the barrel toward the thumb-support member.

2. The aspiration syringe of claim 1, wherein the syringe housing and the plunger are molded together in an integral piece such that the proximal portion of the syringe housing is integral with the proximal portion of the plunger.

3. The aspiration syringe of claim 1, wherein the syringe housing and the plunger are separately molded pieces coupled together in a coupled piece such that the proximal portion of the syringe housing is bonded or adhered to the proximal portion of the plunger.

4. The aspiration syringe of claim 1, wherein the syringe housing includes a longitudinal gap within which the thumb-support member moves relative to the syringe housing when the syringe housing is slid over the barrel.

5. The aspiration syringe of claim 1, wherein the flange includes a pair of finger recesses in a distal face of the flange configured to respectively cradle a pair of finger pads of a pair of fingers of the single hand, the pair of finger recesses configured to encourage placement of each finger of the pair of fingers on an opposite side of the barrel.

6. The aspiration syringe of claim 1, wherein the flange is annular or the flange is coupled to a distal annular member with the flange or the distal annular member encircling the barrel, the flange or the distal annular member configured to accommodate a pair of finger pads of a pair of fingers of the single hand placed against a distal side thereof in any chosen placement of each finger of the pair of fingers around the flange or the distal annular member.

7. The aspiration syringe of claim 6, wherein the flange or the distal annular member includes an annular finger recess in a distal face thereof, the annular finger recess configured to cradle the pair of finger pads of the pair of fingers.

8. The aspiration syringe of claim 1, wherein the thumb-support member includes a single thumb recess in a proximal face of the thumb-support member configured to cradle a thumbpad of a thumb, the single thumb recess configured to encourage approximately longitudinal alignment of the thumb with a surface normal of a surface of the barrel.

9. The aspiration syringe of claim 1, wherein the thumb-support member is coupled to or formed with a proximal annular member with the proximal annular member encircling the barrel, the proximal annular member configured to accommodate a thumbpad of a thumb placed against a proximal side thereof in any chosen placement of the thumb around the proximal annular member.

10. The aspiration syringe of claim 9, wherein the proximal annular member includes an annular thumb recess in a proximal face thereof, the annular thumb recess configured to cradle the thumbpad of the thumb.

11. The aspiration syringe of claim 1, wherein a distal portion of the barrel terminates with a Luer-tapered syringe tip.

12. The aspiration syringe of claim 11, wherein the distal portion of the barrel further terminates with an internally threaded connector around the syringe tip configured to screw together with an externally threaded or flanged connector of another medical device.

13. A method of cannulating with an aspiration syringe, comprising:
obtaining the aspiration syringe, the aspiration syringe including:
a thumb-support member coupled to a proximal portion of a barrel; and
a syringe housing slidably disposed around the barrel including a proximal portion coupled to a proximal portion of a plunger and a distal portion terminating with a flange incorporated into a finger-support member;
inserting a needle coupled to the aspiration syringe into subcutaneous tissue of a patient; and
drawing a vacuum with the aspiration syringe while inserting the needle into the subcutaneous tissue of the patient, the drawing of the vacuum performed with a single hand over a medial portion of the aspiration syringe by squeezing together the finger-support member and the thumb-support member, thereby proximally sliding the syringe housing over the barrel and withdrawing the plunger therefrom.

14. The method of claim 13, further comprising coupling a needle to the aspiration syringe, the coupling including screwing a flanged connector of a needle hub into an internally threaded connector around a syringe tip of the aspiration syringe.

15. The method of claim 13, wherein the squeezing together of the finger-support member and the thumb-support member includes moving the thumb-support member through a longitudinal gap within the syringe housing.

16. The method of claim 13, further comprising placing a pair of finger pads of a pair of fingers of the single hand into a pair of finger recesses in a distal face of the flange on opposite sides of the barrel before drawing the vacuum with the aspiration syringe.

17. The method of claim 13, further comprising placing a pair of finger pads of a pair of fingers of the single hand anywhere against a distal side of the flange when the flange is annular or a distal side of a distal annular member when the flange is coupled thereto, the placing of the pair of finger pad against the distal side of the flange or the distal annular member before drawing the vacuum with the aspiration syringe.

18. The method of claim 17, wherein placing the pair of finger pads anywhere against the distal side of the flange or the distal annular member includes placing the pair of finger pads in an annular finger recess in a distal face of the flange or a distal face of the distal annular member.

19. The method of claim 13, further comprising placing a thumbpad of a thumb of the single hand into a single thumb recess in a proximal face of the thumb-support member before drawing the vacuum with the aspiration syringe, the thumb approximately longitudinally aligned with a surface normal of a surface of the barrel.

20. The method of claim 13, further comprising placing a thumbpad of a thumb of the single hand anywhere against a proximal side of a proximal annular member coupled to or formed with the thumb-support member, the placing of the thumbpad against the proximal side of the proximal annular member before drawing the vacuum with the aspiration syringe.

21. The method of claim 20, wherein placing the thumbpad anywhere against the proximal side of the proximal annular member includes placing the thumbpad in an annular thumb recess in a proximal face of the proximal annular member.

22. The method of claim 13, further comprising establishing a needle tract from a skin surface of the patient to a fluid-containing space within the patient, the establishing of the needle tract realized upon receiving tactile feedback resulting from a pressure differential occurring with a release of the vacuum when a tip of the needle enters the fluid-containing space.

23. The method of claim 13, wherein the fluid-containing space is a blood-vessel lumen including blood as a fluid therein.

24-33. (canceled)
